(19) United States
(12) Patent Application Publication
(10) Pub. No.: US 2017/0166979 A1
(43) Pub. Date: Jun. 15, 2017

(54) PERSONALIZED TREATMENT OF CANCER USING FGFR INHIBITORS

(71) Applicants: Roman K. Thomas, Cologne (DE); Florian Malcher, Cologne (DE)

(72) Inventors: Roman K. Thomas, Cologne (DE); Florian Malcher, Cologne (DE)

(21) Appl. No.: 15/424,900

(22) Filed: Feb. 6, 2017

Related U.S. Application Data
(63) Continuation of application No. 14/716,883, filed on May 20, 2015, now abandoned.
(60) Provisional application No. 62/001,046, filed on May 20, 2014.

(51) Int. Cl. C12Q 1/68; A61K 31/517; A61K 31/53; A61K 31/4025; A61K 31/438; C07K 16/30; A61K 31/737; A61K 31/496; A61K 31/47; C07K 14/705; C12N 15/113

(52) U.S. Cl. CPC C12Q 1/6886; C07K 14/705; A61K 31/517; A61K 31/53; A61K 31/4025; C12N 15/1138; C07K 16/30; A61K 31/737; A61K 31/496; A61K 31/47; A61K 31/438; C12N 2310/11; C07K 2319/30; C07K 2319/40; C12Q 2600/106; C12Q 2600/158

(57) ABSTRACT

The present invention relates to a method for predicting the responsiveness of cancer cells to FGFR1 inhibitors, which comprises the evaluation of the status of FGFR1 gene and the status of MYC. A kit useful for carrying out the method is also provided. In addition, a method of treating cancer such as lung cancer is also provided which includes determining the status of FGFR1 gene and the status of MYC gene, and administering to the cancer patient an FGFR1 inhibitor if the tumor tissue or cells exhibit an increased expression or amplification of the FGFR1 gene, as well as an increased expression or amplification of the MYC gene.

### CROSS-REFERENCE TO RELATED APPLICATIONS

This application is the continuation application of U.S. application Ser. No. 14/716,883 filed on May 20, 2015 which claims the priority of U.S. Provisional Application No. 62/001,046 filed on May 20, 2014, the entire content of which is incorporated herein by reference.

### FIELD OF THE INVENTION

The present invention generally relates to cancer therapy, and particularly to personalized treatment of cancer with an FGFR1 inhibitor based on specific biomarkers.

### BACKGROUND OF THE INVENTION

Oncogenic protein kinases are frequently potential targets for cancer treatment. Examples include ERBB2 amplification in breast cancer, associated with clinical response to antibodies targeting ERBB2 (see Slamon, et al., N. Engl. J. Med., 344, 783-792 (2001)), and KIT or PDGFRA mutations in gastrointestinal stromal tumors, which lead to sensitivity to the KIT/ABL/PDGFR inhibitor imatinib (see Heinrich et al., J. Clin. Oncol., 21, 4342-4349 (2003)). In lung adenocarcinoma, patients with EGFR-mutant tumors experience tumor shrinkage and prolongation in progression-free survival when treated with EGFR inhibitors. See Pao et al., Proc Natl Acad Sci USA 101, 13306-13311 (2004); Paez et al., Science 304, 1497-1500 (2004); Lynch et al., N. Engl. J. Med., 350, 2129-2139 (2004); Mok, et al., N. Engl. J. Med. 361, 947-957 (2009). Furthermore, EML4-ALK gene fusion-positive lung cancers can be effectively treated with ALK inhibitors. Soda et al., Nature 448, 561-566 (2007); Kwak et al., N Engl J Med 363, 1693-1703).

FGFR1 has also been proved to be a target amenable for targeted therapy in a variety of cancer types including breast cancer and bladder cancer. In particular, Weiss, et al. Sci Transl Med 2, 62ra93 (2010) discovered FGFR1 to be the first “druggable” target in squamous-cell lung cancer patients, and frequent and focal FGFR1 gene amplification may serve as the predictor of the effect of FGFR1 inhibitors in causing apoptosis of lung cancer cells. This was the first tractable companion diagnostic marker discovered in squamous cell lung cancer.

However, both in vitro experiments and early stage clinical trials showed that not all cancer cells with FGFR1 gene amplification respond to FGFR1 inhibitors. Therefore, there is still need for additional biomarkers for further refined personalized treatment with FGFR1 inhibitors.

### SUMMARY OF THE INVENTION

The inventors have surprisingly discovered that patients with FGFR1-amplified tumors respond to FGFR1 inhibitors particularly well when the tumors also overexpress the MYC gene.

Accordingly, the present invention provides a method of predicting a patient's response to FGFR1 inhibitors. The method includes the steps of selecting a patient having cancer, such as lung cancer (particularly squamous cell lung cancer), breast cancer, bladder cancer, oral squamous cell carcinoma, esophageal squamous cell carcinoma, ovarian cancer, prostate cancer and renal cancer, determining in tumor cells or tissue obtained from the patient, the presence or absence of FGFR1 gene amplification or gene overexpression, and determining the status of MYC gene amplification or MYC gene expression in tumor cells or tissue obtained from the patient, wherein the detection of both (1) FGFR1 gene amplification or increased FGFR1 gene expression, and (2) MYC gene amplification or increased MYC gene expression would indicate that the patient has an increased likelihood of response to FGFR1 inhibitors, and wherein the absence of (1) or (2) or both would indicate that the patient is less likely to respond to an FGFR1 inhibitor.

In another aspect, the present invention provides a method of predicting a cancer patient's response to FGFR1 inhibitors wherein the patient's cancer cells harbor FGFR1 amplification or overexpression. The method includes the steps of selecting a patient having cancer with FGFR1 amplification or overexpression, and determining the status of MYC gene amplification or MYC gene expression in tumor cells or tissue obtained from the patient, wherein the detection of MYC gene amplification or increased MYC gene expression would indicate that the patient has an increased likelihood of response to FGFR1 inhibitors, and wherein the absence of MYC gene amplification or increased MYC gene expression would indicate that the patient is less likely to respond to an FGFR1 inhibitor.

In another aspect, the present invention provides a method of treating cancer such as lung cancer (particularly squamous cell lung cancer), breast cancer, bladder cancer, oral squamous cell carcinoma, esophageal squamous cell carcinoma, ovarian cancer, prostate cancer and renal cancer, comprising the steps of determining in cancer cells or tissue obtained from the patient, the presence or absence of FGFR1 gene amplification or FGFR1 gene overexpression, and the presence or absence of MYC gene amplification or MYC gene overexpression, and administering to the patient an effective amount of an FGFR1 inhibitor when FGFR1 gene amplification or FGFR1 gene overexpression is detected and MYC gene amplification or MYC gene expression is also detected.

In another aspect, a diagnostic kit consisting in a compartmentalized container, essentially of a first nucleic acid primer or probe that hybridizes to the FGFR1 gene, or a first antibody selectively immunoreactive to FGFR1 protein; and a second nucleic acid primer or probe that hybridizes to the MYC gene, or a second antibody selectively immunoreactive to c-MYC protein. The kit optionally further includes reagents useful for PCR or sequencing or immunoassays.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF THE DRAWINGS

FIG. 1. NIH3T3 cells were retrovirally (pBabe) (co)-transduced with FGFR1 and eight further cancer genes. Colony formation in a 21-day soft agar assay was compared with empty vector controls by the Benjamini-Hochberg corrected t test and classified into strong (++), mild (+; <10 colonies per well), and no (0) transformation. NIH3T3 cells did not survive transduction with MYC alone (X). *, the Benjamini-Hochberg correction is not significant.

FIG. 2. Protein expression and phosphorylation of transduced cells were analyzed by immunoblotting (top). Mesenchymal FGFR1α (full length) could be differentiated from FGFR1β by protein size. Relative colony counts of a 21-day soft agar assay were compared by the Benjamini-Hochberg corrected t test (bottom). Error bars display SD of average counts of three independent experiments.

FIG. 3. Induction of apoptosis (Annexin-V/PI, flow cytometry) in NIH3T3 cells, (co-) transduced with FGFR1β+/−MYC, by 72-hour FGFR inhibition (PD173074, 1 μmol/L). FGFR-dependent H1581 cells (PD173074, 1 μmol/L) as well as ALK-dependent NIH3T3-EML4-ALK

cells (TAE684, 1 μmol/L) were used as positive controls. Resistant HCC15 and NIH3T3-e.V. cells served as negative controls. *, Significant induction of apoptosis.

FIG. 4. Nude mice, engrafted with retrovirally transduced NIH3T3 cells, received BGJ398 (15 mg/kg, q.d., lower curve) or 5% glucose (upper curve), respectively, upon formation of palpable tumors. Volumes of tumors formed by NIH3T3-FGFR1f3 cells (top) and NIH3T3-FGFR1β-MYC cells (bottom) were assessed every second day and compared by the t test. Error bars display SD of three independent experiments.

FIG. 5. MYC was expressed at much higher nuclear levels in the double-transduced cells, which was subject to FGFR-dependent regulation.

FIG. 6. FGFR1-amplified H1581, DMS114, and HCC95 cells as well as HCC15 (NRAS mut) controls were treated with PD173074 (1 μmol/L, 24 hours). Expression levels of MYC, cyclin D1, and actin as well as ERK phosphorylation were monitored by immunoblotting. Con.: positive control NIH3T3-FGFR1β cells.

FIG. 7. Protein expression of MYC was silenced by stable lentiviral transduction of FGFR dependent H1581 cells as well as HCC15, H2882, and HCC95 controls. Knockdown efficiency was validated by immunoblotting for H1581, H2882, and HCC15 cells (top). FGFR dependency was determined by measuring cellular ATP content after 96 hours (bottom).

FIG. 8. Rrelative RNA expression levels of FGFR1-4 (black, blue, green, gray) and MYC (red) in a cohort of 14 cancer cell lines enriched for FGFR1 amplification. Correlation of FGFR dependency and FGFR1×MYC expression levels (inset). Significance of correlation was derived from Student t distribution.

FIG. 9. Segregation of FGFR1 amplification with RNA expression levels of MYC Cancer cell lines were divided into an FGFR-dependent (H1581, DMS114, and HCC1599) GI 50<500 nmol/L, PD173074) versus resistant group (A427, H520, H1703, HCC15, H358, HCC95, H187, SW1271, H526, and DMS153 cells). Expression levels were compared by the Student t test. wt, wild-type.

FIG. 10. Enrichment of FGFR1 phosphorylation, independence of MYC expression in a cohort of 86 FGFR1 -amplified lung cancer patients. Tumor biopsies were analyzed by FGFR1 FISH and stained for MYC expression as well as FGFR1 phosphorylation. Frequencies of positive stains were compared by the Fisher exact test.

FIG. 11. Pathologic examination of a squamous cell tumor biopsy of the BGJ398 responder [BGJ398 trial]. The sample was scored (degrees 0-3) by FGFR1 dual-color FISH (top, normalized copy-number ratio) as well as MYC IHC (bottom, nuclear staining intensity).

FIG. 12. IHC-Scoring of FGFR phosphorylation and MYC expression exhibits enriched

FGFR phosphorylation and variant MYC expression. Phospho-FGFR (top) and MYC (bottom) IHC stains were scored from 0 to 3. A representative sample is shown for each score.

FIG. 13. Fused scans of positron emission tomography (PET) and computer tomography (CT) before (top left, baseline) and after the beginning of BGJ389 therapy (top right, 4 weeks). Baseline CT scan (bottom left); CT after 8 weeks (bottom right) of BGJ398 therapy, showing tumor regression. Target lesions for evaluation of tumor response are highlighted by red arrows. IHC, immunohistochemistry.

FIG. 14. Focality of the 8p12 amplicon as assessed by Deep Cap Analysis Gene a) Expression (CAGE) Sequencing. DNA was extracted from the formalin-fixed tumor sample and cloned into a CAGE sequencing library. Genes of the 8p12 amplicon were enriched in the CAGE chip design. Copy number was inferred from gene coverage and mapped to genomic positions of the Hg18 annotation. b) Pathological examination of a tumor biopsy of the pazopanib responder before therapy. After diagnosis SQLC histology (top left), the sample was scored (degrees 0-3) by FGFR1 FISH (top left middle), phospho-FGFR1 IHC (top right middle) as well as nuclear staining of MYC IHC (top right). Dual color FISH was performed with FGFR1 (green) and CEN8 (red, centromere) probes in order to derive a normalized copy number ratio for FGFR1 amplification. Baseline computer tomographic (CT) scan with tumor in the left lung (bottom left); CT after 4 weeks (bottom middle) and 8 weeks (bottom right) of pazopanib, showing tumor regression with cavitation. Target lesions for evaluation of tumor response are highlighted by red arrows.

### DETAILED DESCRIPTION OF THE INVENTION

Previously it was discovered that FGFR1 inhibitors inhibit growth and induce apoptosis in those cancer cells carrying amplified FGFR1 or with FGFR1 overexpression. However, still many cell lines and tumors with amplified FGFR1 are resistant to FGFR1 inhibitors. The inventors now have surprisingly discovered that in cell lines and tumors with amplified FGFR1, response to FGFR1 inhibitors are correlated with MYC expression. That is, FGFR1 gene amplification or overexpression together with MYC gene amplification or overexpression give rise to much greater predictive power for response to FGFR1 inhibitors than FGFR1 status alone, and thus could lead to better refinement of patient selection for personalized treatment with FGFR1 inhibitors.

Accordingly, the present invention provides a method of predicting a cancer patient's response to FGFR1 inhibitors. The method includes the steps of selecting a patient having cancer such as lung cancer (particularly squamous cell lung cancer, small cell lung cancer, etc.), breast cancer, bladder cancer, oral squamous cell carcinoma, esophageal squamous cell carcinoma, ovarian cancer, prostate cancer and renal cancer, determining in cancer cells or tissue obtained from the patient, the presence or absence or status of focal FGFR1 gene amplification or FGFR1 gene overexpression, as well as the presence or absence or status of MYC gene amplification or MYC gene overexpression, wherein the presence of focal FGFR1 gene amplification or FGFR1 overexpression as well as MYC gene amplification or MYC gene overexpression would indicate that the patient has an increased likelihood of response to FGFR1 inhibitors. The absence of focal FGFR1 gene amplification or overexpression, and/or MYC gene amplification or MYC gene overexpression would indicate that the patient is less likely to respond to FGFR1 inhibitors.

In another aspect, the present invention provides a method of predicting a cancer patient's response to FGFR1 inhibitors. The method includes the steps of selecting a cancer patient whose cancer cells or tumor tissue is determined to harbor FGFR1 gene amplification or FGFR1 gene overexpression, and determining in cancer cells or tissue obtained from the patient, the presence or absence or status of MYC gene amplification or MYC gene overexpression, wherein the presence of MYC gene amplification or MYC gene overexpression would indicate that the patient has an increased likelihood of response to FGFR1 inhibitors. The absence of MYC gene amplification or MYC gene overexpression would indicate that the patient is less likely to respond to FGFR1 inhibitors. In some embodiments, the patients may have lung cancer (particularly squamous cell lung cancer, small cell lung cancer, carcinoid, etc.), breast cancer, bladder cancer, oral squamous cell carcinoma, esophageal squamous cell carcinoma, ovarian cancer, prostate cancer or renal cancer.

In another aspect, the present invention provides a method of predicting a cancer patient's response to an FGFR1 inhibitor. The method includes the steps of selecting a cancer patient whose cancer cells or tumor tissue is determined to harbor MYC gene amplification or MYC gene overexpression, and determining in cancer cells or tissue obtained from the patient, the presence or absence or status of FGFR1 gene amplification or FGFR1 gene overexpression, wherein the presence of FGFR1 gene amplification or FGFR1 gene overexpression would indicate that the patient has an increased likelihood of response to FGFR1 inhibitors. The absence of FGFR1 gene amplification or FGFR1 gene overexpression would indicate that the patient is less likely to respond to an FGFR1 inhibitor. In some embodiments, the patients may have lung cancer (particularly squamous cell lung cancer, small cell lung cancer, carcinoids etc.), breast cancer, bladder cancer, oral squamous cell carcinoma, esophageal squamous cell carcinoma, ovarian cancer, prostate cancer or renal cancer.

In another aspect, the present invention provides a method of treating cancer. The method includes determining the presence or absence or status of focal FGFR1 gene amplification or FGFR1 gene overexpression and the presence or absence or status of MYC gene amplification or MYC gene overexpression in cancer cells or tissue obtained from the patient. The determined status of focal FGFR1 gene amplification or FGFR1 gene overexpression, and status of MYC gene amplification or MYC gene overexpression may be used to guide the treatment decision for the patient. Specifically, a therapeutically effective amount of an FGFR1 inhibitor is administered if focal FGFR1 gene amplification or FGFR1 gene overexpression as well as MYC gene amplification or MYC gene overexpression are detected or present. Thus, the treatment method may also include a step of administering a therapeutically effective amount of an FGFR1 inhibitor in the presence of focal FGFR1 gene amplification or FGFR1 gene overexpression, and the presence of MYC gene amplification or MYC gene overexpression. When focal FGFR1 gene amplification (or FGFR1 gene overexpression) and/or MYC gene amplification (or MYC gene overexpression) is absent in the cancer cells or tissue, the patient may be administered a treatment regimen free of FGFR1 inhibitors. In preferred embodiments, FGFR1 gene amplification status is determined. In preferred embodiments, the patient has lung cancer (particularly squamous cell lung cancer, small cell lung cancer, or lung carcinoid), breast cancer, bladder cancer, oral squamous cell carcinoma, esophageal squamous cell carcinoma, ovarian cancer, prostate cancer, or renal cancer.

In another aspect, the present invention provides a method of treating cancer in a patient with cancer cells or tissue determined as having FGFR1 gene amplification or FGFR1 gene overexpression. The method comprises determining the presence or absence or status of MYC gene amplification or MYC gene overexpression in cancer cells or tissue obtained from the patient, and administering a therapeutically effective amount of an FGFR1 inhibitor to the patient. Particularly, a therapeutically effective amount of an FGFR1 inhibitor to the patient if MYC gene amplification or MYC gene overexpression is detected. In preferred embodiments, the cancer to be treated is lung cancer (particularly squamous cell lung cancer, small cell lung cancer, or lung carcinoid), breast cancer, bladder cancer, oral squamous cell carcinoma, esophageal squamous cell carcinoma, ovarian cancer, prostate cancer, or renal cancer.

In another aspect, the present invention provides a method of treating cancer in a patient with cancer cells or tissue determined as having MYC gene amplification or MYC gene overexpression. The method comprises determining the presence or absence or status of FGFR1 gene amplification or FGFR1 gene overexpression in cancer cells or tissue obtained from the patient, and administering a therapeutically effective amount of an FGFR1 inhibitor to the patient. Particularly, a therapeutically effective amount of an FGFR1 inhibitor to the patient if FGFR1 gene amplification or FGFR1 gene overexpression is detected. In preferred embodiments, the cancer to be treated is lung cancer (particularly squamous cell lung cancer, small cell lung cancer, or lung carcinoid), breast cancer, bladder cancer, oral squamous cell carcinoma, esophageal squamous cell carcinoma, ovarian cancer, prostate cancer, or renal cancer.

Thus, in preferred embodiments, the method is employed to treat lung cancer, in particular squamous cell lung cancer, which comprises identifying a patient having, or diagnosing a patient as having, squamous cell lung cancer; determining the status of focal FGFR1 gene amplification or FGFR1 gene overexpression in squamous cell lung cancer cells or tissue obtained from the patient; determining the status of MYC gene amplification or MYC gene overexpression in squamous cell lung cancer cells or tissue obtained from the patient; and administering a therapeutically effective amount of an FGFR1 inhibitor to the patient.

In other preferred embodiments, the method is employed to treat lung cancer, in particular squamous cell lung cancer, which comprises identifying a patient having, or diagnosing a patient as having, squamous cell lung cancer; determining the status of focal FGFR1 gene amplification or FGFR1 gene overexpression in squamous cell lung cancer cells obtained from the patient, and determining the status of MYC gene amplification or MYC gene overexpression in squamous cell lung cancer cells obtained from the patient; and administering a therapeutically effective amount of an FGFR1 inhibitor to the patient when (1) focal FGFR1 gene amplification or FGFR1 gene overexpression and (2) MYC gene amplification or MYC gene overexpression are both detected in the squamous cell lung cancer cells obtained from the patient. To put it differently, the method comprises administering a therapeutically effective amount of an FGFR1 inhibitor to a patient diagnosed of squamous cell lung cancer and with tumor cells determined to have focal FGFR1 gene amplification or FGFR1 gene overexpression as well as MYC gene amplification or MYC gene overexpression.

In other preferred embodiments, the method of treating lung cancer, in particular squamous cell lung cancer, comprises identifying a patient having, or diagnosing a patient as having, squamous cell lung cancer; determining the status of focal FGFR1 gene amplification or FGFR1 gene overexpression, and MYC gene amplification or MYC gene overexpression, in squamous cell lung cancer cells obtained from the patient; and when (1) focal FGFR1 gene amplification or FGFR1 protein overexpression and/or (2) MYC gene amplification or MYC gene overexpression is absent in the squamous cell lung cancer cells, administering to the patient a treatment regimen free of FGFR1 inhibitors.

MYC (v-myc avian myelocytomatosis viral oncogene homolog, Gene ID: 4609), also known as c-myc, functions as a transcription factor that regulates transcription of specific target genes.

In the methods of the present invention, focal gene amplification is determined to be present when greater than 2 copies of the genomic DNA of a particular gene (e.g., FGFR1 or MYC) are detected in a cancer cell from a cancer patient, e.g., as measured by FISH, CISH, real-time PCR, sequencing or microarray. In preferred embodiments, focal gene amplification is determined to be present when at least 4, 7 or 8, or more preferably at least 9 chromosomal copies of a gene (FGFR1 or MYC) are detected in a cancer cell from a cancer patient, e.g., as measured by FISH, CISH, real-time PCR, sequencing or microarray. The status of FGFR1 or MYC gene amplification means the presence, absence or the degree of amplification of the FGFR1 or MYC gene.

Focal gene amplification or polysomy can be detected using any method known in the art. Specifically, the FGFR1 gene is located in chromosome 8 at about 8p11.2-p11.1, or about 8p11.23 to 8p11.22, such as the 133 kb region (chr8:38436349-38569287) including the FGFR1 gene as well as FLJ43582 gene. Thus, focal FGFR1 amplification or polysomy can be detected by directly measuring the copy number of the FGFR1 gene itself per cell, or by indirectly measuring any amplification of at least part of the 133 kb region (chr8:38436349-38569287), or amplification of the FLJ43582 gene. The MYC gene is located in chromosome 8q24, and has been found to be amplified in different cancer types including lung cancer, particularly squamous cell lung cancer.

A variety of techniques are known in the art suitable for detecting gene amplification (or increase of genomic DNA copy numbers per cell), mRNA overexpression or protein overexpression, in a tissue or cell sample. For example, in situ hybridization using nucleic acid probes can be performed using any appropriate technique, such as fluorescence in situ hybridization (FISH) (e.g., interphase, metaphase, or fiber FISH), and chromogenic in situ hybridization (CISH) to detect gene amplification or gene copy changes at the chromosome level. See Pinkel et al. Proc Natl Acad Sci USA, 85:9138-42 (1988); Sholl et al., Mod. Pathol., (10):1028-35 (2007). Generally, labeled single-stranded nucleic acid probes can be contacted with a tissue or cell sample (e.g., fresh-frozen or FFPE tumor samples) under conditions such that the probes hybridize to the genomic region of interest in cells, and the hybrids are then detected by, e.g., fluorescence signal or enzymatic detection. For example, FGFR1 CISH may be performed with FGFR1 ZytoDot-SPEC Probe (Zytovision GMbH, Bremerhaven, Germany) and the SPoT-Light CISH Polymer Detection Kit (Invitrogen). See Turner et al., Cancer Res., 70(5); 2085-94 (2010). FISH probe for MYC is also commercially available as “MYC/CEN-8 FISH Probe Mix” from Dako.

Alternatively, the multiplex ligation-dependent probe amplification (MLPA) may also be used to detect gene amplification or genomic copy number variation. See e.g., Villamón et al., Histol. Histopathol., 26, 343-350 (2011); Kozlowski et al., Electrophoresis, (23):4627-36 (2008), all of which being incorporated herein by reference.

Other suitable methods known in the art also include SNP genomic array, genomic hybridization to cDNA microarrays, comparative genomic hybridization (CGH), oligonucleotide array CGH, and spectral karyotyping (SKY). See U.S. Pat. No. 7,424,368; Heiskanen, et al., Cancer Res., 60:799 (2000); Kallioniemi et al., Comparative Genomic Hybridization: A Powerful New Method for Cytogenetic Analysis of Solid Tumors, Science, 258:818-821 (1992); Pinkel et al., High-Resolution Analysis of DNA Copy Number Variation Using Comparative Genomic Hybridization to Microarrays, Nat. Genet., 20:207-211 (1998); Schrock, et al., Science, 273:494-7 (1996)), all of which being incorporated herein by reference. Additionally, gene amplification may also be detected using next-generation sequencing by comparing the number of sequence reads in non-overlapping windows between patient and control samples. See e.g., Hayes et al., Genomics, 102(3):174-181 (2013)

A preferred method for detecting gene amplification is genomic DNA-based quantitative real-time PCR or qPCR. See Konigshoff et al., Clinical Chemistry 49: 219-229, 2003, which is incorporated herein by reference. The target DNA to be assayed may be amplified in real-time PCR by, e.g., conventional techniques such as TaqMan, Scorpion, molecular beacons, and the amount of amplified DNA product may be detected by non-sequence specific fluorescence dyes (e.g. SybrGreen), or labeled probes such as TaqMan probes, FRET probes, and molecular beacons. See Bartlett and Stirling, PCR Protocols, in Methods in Molecular Biology, 2^{nd} ed., 2003, Humana Press, Totowa, N.J., USA. For copy analysis, an exogenous DNA standard or endogenous housekeeping gene or DNA sequence can be used as a reference, as is known in the art.

In the context of the above and below description of the present invention, the gene expression of FGFR1 or MYC means the gene expression level of the FGFR1 or MYC gene as measured by any suitable methods. Typically, the level of expression of a particular gene may be reflected at the transcription level by measuring the level of mRNA transcribed from the FGFR1 or MYC gene in a cell or tissue, or at the translation level by measuring the protein level in a cell or tissue.

Quantitative real-time PCR is particularly suitable for determining a particular mRNA level in a cell or tissue sample, in which case mRNA is first reverse transcribed into cDNA, which is then amplified by PCR using gene-specific oligonucleotide PCR primers. This qRT-PCR method is well-known in the art. Next-generation sequencing or microarray may also be used for detecting mRNA levels. Additionally, in situ hybridization may also be used to detect in situ the mRNA level of the FGFR1 or MYC gene in a cell or tissue sample, e.g., in a FFPE tissue sample.

For detecting the FGFR1 or MYC protein expression in a tumor cell or tissue sample, any known methods for measuring protein level in cells or tissue samples may be used for the present invention. Examples of such methods include, but are not limited to, immunohistochemistry (IHC), ELISA, Western blot, protein microarray, etc. Typically an antibody specifically immunoreactive with FGFR1 or MYC protein is contacted with a cell or tissue sample under conditions to allow immunoreaction with FGFR1 or MYC proteins in the sample, and the amount of bound antibody is measured. In IHC analysis, typically an FFPE tumor sample may be used. For ELISA, Western blot and protein microarray analysis, the samples may be FFPE samples or fresh frozen samples, and are preferably homogenized and extracted before contact with an FGFR1 or MYC antibody, as is generally known in the art.

In preferred embodiments, the presence or absence of focal FGFR1 or MYC gene amplification in a cancer cell or tissue obtained from a patient is determined by a process comprising nucleic acid hybridization, e.g., in situ hybridization analysis, or real-time PCR or next-generation sequencing.

In other preferred embodiments, the presence or absence of FGFR1 or MYC mRNA overexpression in cancer cell or tissue obtained from a patient, is determined by qRT-PCR or microarray analysis or in situ RNA detection or RNA sequencing.

In other preferred embodiments, the presence or absence of FGFR1 or MYC protein overexpression in a cancer cell or tissue obtained from a patient, is determined by IHC.

As is already clear from the above, a sample to be tested by the methods of the present invention may be one or more cancer cells (e.g., circulating free tumor cells), or cancer tissues (fresh, fresh frozen, or FFPE samples).

FGFR1 inhibitors applicable to the methods of the presentation are generally known in the art, and are all characterized by significantly inhibiting the kinase activity of the FGFR1 protein, or specifically decreasing the amount of such kinase activity or preventing the activation of such kinase activity in cells. Thus, exemplary FGFR1 inhibitors include, but are not limited to, small organic molecule inhibitors of FGFR1 kinase activity, as well as siRNA and antisense molecules targeting FGFR1 or FGF1 mRNA, antibodies against FGFR1 or FGF1 protein and other molecules capable of antagonizing against FGF signaling through FGFR1. For example, FGF1 traps are considered FGFR inhibitors. Various methods for identifying FGFR1 inhibitors and determining whether a molecule is an FGFR1 inhibitor are generally known in the art, and are disclosed, e.g., in U.S. Pat. Nos. 5,783,683, 6,677,368, and 7,737,149, US Patent Application Publication Nos. 20040014024 and 20100273811, all of which are incorporated herein by reference. It is noted that, for purposes of the present invention, suitable FGFR1 inhibitors may or may not also act upon other targets such as VEGFRs, PDGFR, FGFR2, FGFR3, FGFR4, etc. Indeed, inhibitors of FGFR2, FGFR3 or FGFR4 may also inhibit FGFR1. “FGFR1 inhibitors” is therefore used herein to refer to FGFR1-specific inhibitors as well as a drug that inhibits both FGFR1 and one or more other FGFR proteins.

Examples of small molecule FGFR1 inhibitors known in the art include those disclosed in, e.g., U.S. Pat. Nos. 6,677,368, 6855730, 7,528,142, 7109219, and US Patent Application Publication Nos. 20040014024, 20050209247, 20080004302, 20080153812, 20090318468, 20100120761, 20100286209, and PCT Publication No. WO2002022598, all of which being incorporated herein by reference. Specific examples of commonly known FGFR1 inhibitors include, cediranib, brivanib (Bristol-Myers Squibb), TSU-68 (Teiho), BIBF1120 (Boehringer Ingelheim), dovitinib (Novartis), Ki23057, MK-2461, E7080 (Eisai), PD173074, SU5402, BGJ398 (Novartis), E-3810 (Ethical Oncology Science), AZD4547 (AstraZeneca), and PLX052, etc. Examples of antisense molecules targeting FGFR1 mRNA are disclosed in U.S. Pat. No. 5,783,683, which is incorporated herein by reference. Examples of FGFR1-targeting antibodies are disclosed in U.S. Pat. No. 7,498,416, which is incorporated herein by reference. A fusion protein that exhibits inhibitory effect on FGFR1 is disclosed in U.S. Pat. No. 7,678,890, which is incorporated herein by reference. In addition, sulf1-modified heparin compounds useful as FGFR1 inhibitors are also disclosed in US Patent Application Publication No. 20050227921, which is incorporated herein by reference. Methods of administering the FGFR1 inhibitors to patients for treating cancer are also disclosed in the references provided herein.

The methods of the present invention are applicable to all these and other FGFR1 inhibitors. Thus, methods are provided for predicting a cancer patient's response to any one of such inhibitors, based on the FGFR1 gene amplification or gene expression status and the status of MYC gene amplification or gene expression. Methods are also provided for treating cancer using one or more of the FGFR1 inhibitors, which includes determining the FGFR1 gene amplification or gene expression status, determining the MYC gene amplification or gene expression status, and administering such FGFR1 inhibitors according to the status, as described in details above.

The present invention also provides a diagnostic kit for detecting FGFR1 gene amplification or overexpression and MYC gene amplification or gene overexpression in a cell or tissue sample obtained from a patient. The kit may include a compartmentalized carrier for the various components of the kit. The carrier can be a container or support, in the form of, e.g., bag, box, tube, rack, and is optionally compartmentalized. The carrier may define an enclosed confinement for safety purposes during shipment and storage. The kit also includes various components useful in detecting FGFR1 gene amplification or overexpression and MYC gene amplification or gene expression in accordance with the present invention using the above-discussed detection techniques. Thus, for example, the kit may include one or more FISH probes specific to the chromosome region spanning chr8:38436349-38569287, and/or one or more FISH probes bybridizing to the MYC gene, and the probes may be labeled with a tag. Other reagents generally required for FISH analysis may also be included. In other embodiments, the kit may include one or more oligonucleotide chips having, on a solid support, probes capable of hybridizing the FGFR1 and/or MYC gene sequence. In another embodiment, the kit may include a pair of PCR primers useful in amplifying an FGFR1 gene sequence in real time PCR and/or PCR primer pair for amplifying a MYC gene sequence. In addition to the primer pair, the kit may include reagents useful in PCR, e.g., Taq polymerase, PCR buffer, dNTP, etc. The kit may also include a probe such as a TaqMan probe hybridizing to the FGFR1 or MYC gene sequence. In the above embodiments, the probe and oligonucleotides in the detection kit can be labeled with any suitable detection marker including but not limited to, radioactive isotopes, fluorephores, biotin, enzymes (e.g., alkaline phosphatase), enzyme substrates, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977). Alternatively, the probe and oligonucleotides included in the kit are not labeled, and instead, one or more markers are provided in the kit so that users may label the oligonucleotides at the time of use. In still other embodiments, the kit may include an antibody specific to FGFR1 protein and useful in immunoassay (e.g., immunohistochemical analysis) of FGFR1 protein expression in cell or tissue sample from a patient. The kit may also include an antibody specific to the MYC protein and useful in immunohistochemical analysis of MYC protein expression in cell or tissue sample from a patient, as well as reagents useful in IHC, e.g., secondary antibodies, chromogenic dyes, etc. In addition, the detection kit preferably includes instructions on using the kit for detecting FGFR1 gene amplification or overexpression and MYC gene amplification or gene expression in a cell or tissue sample from a obtained from a patient, in accordance with the detailed description above.

In yet another embodiment, the kit may include an antibody specific to a phosphorylated form of FGFR1 protein and useful in immunoassay (e.g., immunohistochemical analysis) of phospho-FGFR1 protein expression in cell or tissue sample from a patient. The kit may also include an antibody specific to the MYC protein and useful in immunohistochemical analysis of MYC protein expression in cell or tissue sample from a patient, as well as reagents useful in IHC, e.g., secondary antibodies, chromogenic dyes, etc.

Thus, the diagnostic kit of the present invention includes in a compartmentalized container: (1) a first component chosen from a nucleic acid probe hybridizing to the FGFR1 gene, a pair of PCR primers useful in amplifying an FGFR1 gene sequence, an antibody specific to FGFR1 protein; and (2) a second component chosen from a nucleic acid probe hybridizing to the MYC gene, a pair of PCR primers useful in amplifying a MYC gene sequence, an antibody specific to the c-MYC protein. The kit may optionally include other components such as enzyme, buffer, dye or label, antibody, etc.

In one embodiment, the diagnostic kit of the present invention comprises in a compartmentalized container: a first pair of PCR primers useful in amplifying a FGFR1 gene fragment or a part of the FGFR1 cDNA sequence, and a second pair of PCR primers useful in amplifying a region of the MYC gene sequence or a part of the MYC cDNA sequence, and optionally a polymerase, PCR buffer, and/or dNTP etc. Optionally, the kit further includes a labeled probe hybridizing to the FGFR1 gene fragment or part of the FGFR1 cDNA sequence, and/or a labeled probe hybridizing to the MYC gene fragment or part of the MYC cDNA sequence.

For example, the kit may include components useful or necessary for real-time PCR amplification of a genomic fragment of the FGFR1 gene, or real-time PCR amplification of a cDNA fragment of the FGFR1 gene, or sequencing the FGFR1 genomic DNA (e.g. by next-gen sequencing), or in situ hybridization (e.g., FISH, CISH, ISH etc.) detection of FGFR1 gene amplification, or immunoassay detection (e.g., IHC, ELISA, etc.) of FGFR1 protein. In the same kit, components may be included useful or necessary for real-time PCR amplification of a genomic fragment of the MYC gene, or real-time PCR amplification of a cDNA fragment of the MYC gene, or sequencing the MYC genomic DNA (e.g. by next-gen sequencing), or in situ hybridization detection (e.g., FISH, CISH, ISH etc.) of MYC gene amplification, or immunoassay detection (e.g., IHC, ELISA, etc.) of c-MYC protein. Such useful components should be apparent to skilled artisans apprised of the present invention.

Typically, once the FGFR1 gene amplification or overexpression and MYC gene amplification or gene expression status is analyzed in a lab, physicians or patients or other researchers may be informed of the result. Specifically the result may be cast in a transmittable form that can be communicated or transmitted to other researchers or physicians or genetic counselors or patients. Such a form can vary and can be tangible or intangible. The result with regard to the presence or absence of in the individual tested can be embodied in descriptive statements, diagrams, photographs, charts, images or any other visual forms. The statements and visual forms can be recorded on a tangible media such as papers, computer readable media such as floppy disks, compact disks, etc., or on an intangible media, e.g., an electronic media in the form of email or website on internet or intranet. In addition, the result may also be recorded in a sound form and transmitted through any suitable media, e.g., analog or digital cable lines, fiber optic cables, etc., via telephone, facsimile, wireless mobile phone, internet phone and the like.

The test result may be received and/or input into a computer system and processed by a computer program product in the computer system, e.g., in a hospital or clinic.

### EXAMPLE

Materials and Methods

Cell Lines

Cancer cell lines, HEK293T and NIH3T3 cells were purchased from American Type Culture Collection and German Resource Centre for Biological Material (DSMZ) and cultured using either RPMI or Dulbecco's Modified Eagle Medium (DMEM) high-glucose media,

supplemented with 10% fetal calf serum (FCS). Adherent cells were routinely passaged by washing with PBS buffer and subsequent incubation in Trypsin/EDTA. Trypsin was inactivated by the addition of culture medium and cells were plated or diluted accordingly.

Suspension cell lines were passaged by suitable dilution of the cell suspension. All cells were cultured at 37° C. and 5% CO₂. The identity of all cell lines included in this study was authenticated by genotyping (SNP 6.0 arrays, Affymetrix) and all cell lines are tested for infection with mycoplasma (MycoAlert, Lonza). Furthermore, the identity of the H1581 cell line was ensured by short tandem repeat profiling (DNA fingerprinting).

Cell Line Stimulation

Cell lines were starved from bovine serum for 24 hours and stimulated by a collection of 6 FGF ligands (1 ng/mL) and heparin (10 μg/mL) for 20 minutes. In addition, the FGFR1 inhibitor PD173074 (1 μmol/L) was added 40 minutes before stimulation by FGF-1 and FGF-2. Phosphorylation of FGFR, ERK, AKT, and the FGFR1 signaling adapter protein FRS2a as well as total expression of ERK and FGFR1 were assessed by immunoblotting.

Whole Transcriptome Sequencing (RNAseq)

Total RNA was extracted from fresh-frozen lung tumor tissue containing at least 60% tumor cells. Depending on the tissue size, 15-30 slides were cut using a cryostat (Leica) at −20° C. Material for RNA extraction was disrupted and homogenized for 2 minutes at 20

Hz by Tissue Lyser (Qiagen). RNA was extracted using the Qiagen RNeasy Mini Kit. RNA quality was assessed by a Bioanalyzer; samples showing an RNA integrity number (RIN)>8 were retained for transcriptome sequencing. We cloned cDNA strands of 250 bp into a sequencing library, allowing us to sequence 95-bp paired-end reads without overlap. All RNAseq libraries were analyzed on the Illumina Genome Analyzer IIx. Gene coverage was used to differentiate splice variants of FGFR1. Mesenchymal splice variants of FGFR1 were differentiated by coverage of exon 2, whereas coverage of tissue-specific exons 8 (IIIb/IIIc)distinguished epithelial (IIIb) from mesenchymal (Mc) forms.

Ouantitative Real Time PCR

Quantitative real-time PCR was performed using a 7300 Real-Time PCR System (Applied Biosystems) and Power SYBR Green PCR Master Mix (Applied Biosystems) with primer pairs specific for GAPDH (QT01192646, Qiagene) (58° C.), MYC (58° C.), FGFR1 (56° C.), FGFR2 (56° C.), FGFR3 (56° C.) and FGFR4 (56° C.). ACt-values were determined using the 7300 System Software (Applied Biosystems) using GADPH as reference control. Gene expression was calculated by AACt-method.

Xenograft Mouse Models

All animal procedures were approved by the local animal protection committee and the local authorities. Transduced NIH3T3 and tumor cells were resuspended in RPMI or DMEM medium and injected (5×10 6 cells per tumor) subcutaneously into the flanks of 8- to 15-week-old male nude mice [Rj:NMRI-nu (nu/nu), Janvier Europe] under 2.5% isoflurane anesthesia.

To assess the effect of FGFR1 inhibitors in vivo, NVP-BGJ 398 (Novartis) was dissolved in a vehicle solution (33% PEG300, 5% glucose) for xenograft application. Tumor size was monitored every second day by measurement of perpendicular diameters by an external caliper and calculated by use of the modified ellipsoid formula [V=½ (Length×Width 2)]. Oral therapy was started when tumors reached a volume of 100 mm 3. Mice received daily either

BGJ398 (15 mg/kg) or vehicle solution. After 14 (NIH3T3 FGFR1(3 +MYC), 16 (NIH3T3 EML4-ALK, KRAS G12V), or 25 (NIH3T3 e.V., FGFR1α/β) days of therapy, respectively, mice were sacrificed by intraperitoneal injection of ketamine/xylazine (300/60 mg/kg).

To examine ligand dependency in vivo, AdCMV-null virus (Vector Biolabs) and AdsFGFR virus (titer: 1×10 10, contributed as a kind gift by Gerhard Christofori, University of Basel) were mixed with tumor cells in DMEM for subcutaneous injection. Tumor formation was

monitored twice a week by careful visual inspection and palpation of the skin. As soon as tumors became palpable, diameters were measured by an external caliper to determine tumor volumes. In addition, animal weights were documented weekly. Eight weeks after injection of H1581 and A549 tumor cells, animals were sacrificed. Subcutaneous tumors as well as livers were resected and fixed in 4% formaldehyde for immunohistochemical staining and virus detection, respectively.

ELISA Assay

Cell culture supernatants were collected, centrifuged (200 rcf, 5 minutes), concentrated by ultracentrifugation units (Satorius AG) and analyzed for FGF concentrations by ELISA (Abcam). In addition, protein was extracted from cells, collected in equal amounts of lysis buffer (Cell Signaling Technology), and measured by Bradford assay (Pierce). Normalized FGF concentrations (c Norm) were derived as ratios of FGF and lysate protein concentrations.

Results

Cell-Autonomous Transformation by FGFR1 and MYC

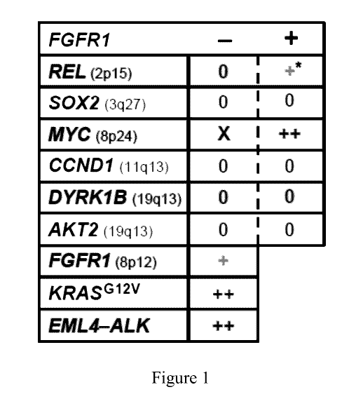

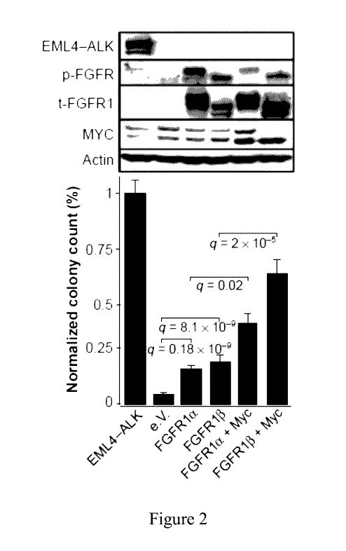

We sought to test whether wild-type FGFR1 was oncogenic when overexpressed and analyzed the oncogenic phenotype of NIH3T3 cells ectopically expressing FGFR1 in soft agar assays. Whole-transcriptome sequencing (RNAseq) of six primary FGFR/-amplified squamous cell lung cancer tumors as well as four amplified cancer cell lines revealed that mesenchymal splice variants of FGFR1 were predominantly expressed in the FGFR1 inhibitor-sensitive cell lines. We therefore cloned these splice variants (FGFR1-IIIc-α, FGFR1-IIIc-β) from H1581 cells and transduced NIH3T3 cells with these variants of FGFR1 either alone or together with six additional genes (REL, SOX2, MYC, CCND1, DYRK1B, AKT2) with a possible role in squamous cell lung cancer biology. The latter genes are located in or close to recurrent amplicons in this lung tumor subtype. Both FGFR1 variants reproducibly induced mild transformation of NIH3T3 cells to anchorage independent growth (q=8×10⁻⁹; FIGS. 1 and 2).

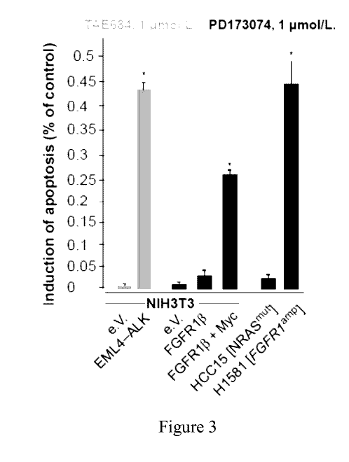

In our hands NIH3T3 cells did not survive transduction with MYC alone. However, transduction of NIH3T3 cells with MYC and FGFR1 (q=2×10⁻⁵) was strongly oncogenic as determined by the number and size of colonies in soft agar (FIG. 2). Similar to FGFR-dependent H1581 cells, treatment with the FGFR1 inhibitor PD173074 induced apoptosis in these FGFR1-MYC cotransduced NIH3T3 cells, but not in cells expressing FGFR1 alone (FIG. 3). Thus, FGFR/-amplified cells coexpressing MYC may be more susceptible to FGFR inhibition, which has been similarly reported for FGFR2-mutant breast cancer.

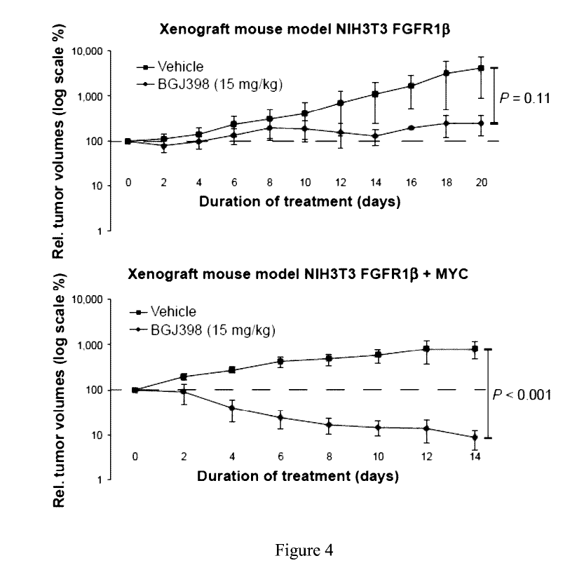

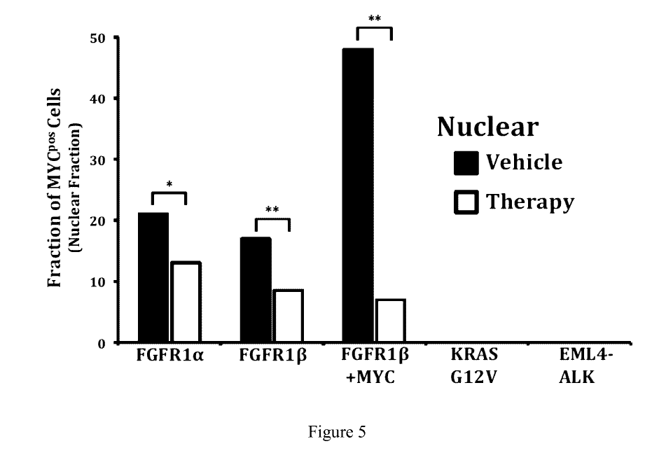

Injection of NIH3T3 FGFR1-IIIc-α and -β cells into nude mice led to palpable subcutaneous tumors after a median of 20 days (FIG. 4, top). HEK293 cells, transduced with FGFR1, similarly induced subcutaneous tumors in vivo, and intravenous injection of NIH3T3 FGFR1 a cells led to tumor growth in the lungs (data not shown). Treatment with the FGFR1 inhibitor BGJ398 (15 mg/kg, q.d.) repressed tumor growth of NIH3T3 cells expressing either of the mesenchymal FGFR1 splice variants (FIG. 4). Thus, the catalytic activity of FGFR1 was required for tumor formation in vivo. However, FGFR inhibition by BGJ398 did not induce tumor shrinkage in tumors expressing FGFR1 alone. In contrast, this treatment led to regressions of tumors coexpressing FGFR1 and MYC (FIG. 3D; P<0.001). Of note, the tumors expressing FGFR1 alone also exhibited low nuclear expression levels of MYC. However, MYC was expressed at much higher nuclear levels in the double-transduced cells, which was subject to FGFR-dependent regulation (FIG. 5). Thus, FGFR1-expressing tumors upregulate MYC in vivo, but only very high levels of MYC expression are likely to govern susceptibility to FGFR inhibition.

FGFR1 Dependency and MYC Expression

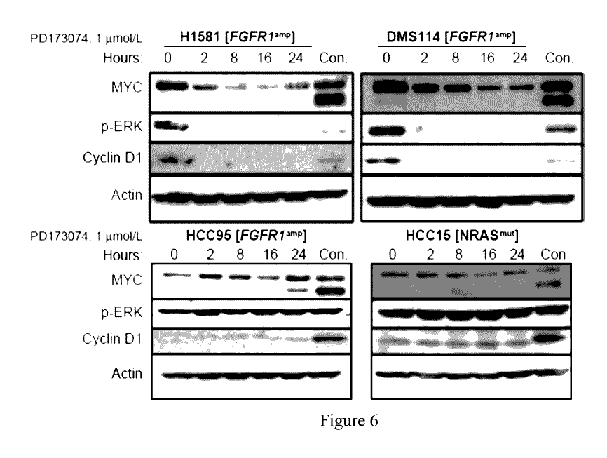

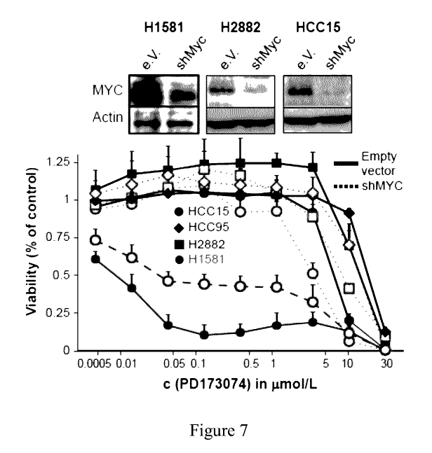

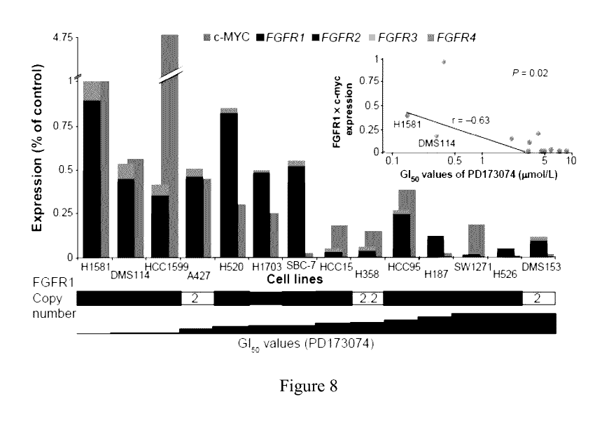

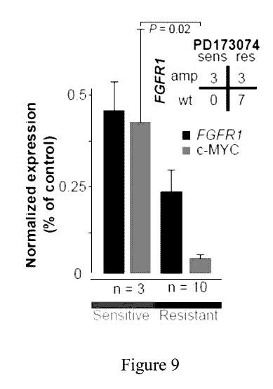

Supporting the notion that MYC may interplay with FGFR1 signaling, we found it to be strongly regulated by FGFR1 in the FGFR-dependent cell lines H1581 and DMS114. Accordingly, levels of MYC and of cyclin D1 decreased upon FGFR inhibition within 24 hours (FIG. 6). In contrast, expression levels remained relatively stable in FGFR1 -amplified HCC95 and H520 cells, which are resistant to FGFR inhibition, as well as in the NRAS-mutant HCC15 cells (FIG. 6). MYC was also highly regulated on the transcriptional level in H1581, but not in H520 cells (data not shown). To formally test whether MYC expression levels dictate sensitivity to FGFR inhibition, we stably silenced MYC in H1581 cells. This manipulation led to FGFR1 inhibitor resistance (FIG. 7). Unfortunately, we could not test this hypothesis in DMS114 cells because they did not tolerate MYC knockdown. We next examined the regulation of downstream effectors in MYC signaling and found that the mitochondrial apoptosis mediators were predominantly affected by FGFR inhibition (PD173074, 1 μmol/L); loss of the mitochondrial membrane potential as well as cytochrome C release occurred robustly after 72 hours in FGFR-dependent cell lines. Further analysis of RNAseq data revealed that tumor samples, in which the amplicon centered on FGFR1, expressed higher levels of MYC (P=0.002) compared with other 8p12-amplified samples. However, we were not able to detect a statistically significant co-occurrence of amplified 8p12 and MYC. Therefore, we analyzed the transcription levels of MYC in our cell line panel (n=14). Levels of MYC gene expression predicted FGFR1 inhibitor sensitivity in individual 8p12-amplified cell lines (P=0.02; FIG. 8) as well as in groups of sensitive versus insensitive cell lines (FIG. 9).

Altogether, we used cell culture and xenograft experiments (FIGS. 2 & 4) to study the interplay of FGFR1 with MYC. In all independent approaches, we observed that MYC modulates oncogenic transformation, cell-autonomous signaling, and FGFR1 inhibitor response in FGFR1-amplified or overexpressing cells (FIGS. 6-9).

Prevalence of MYC Expression in Primary FGFR1-Amplified Lung Tumors

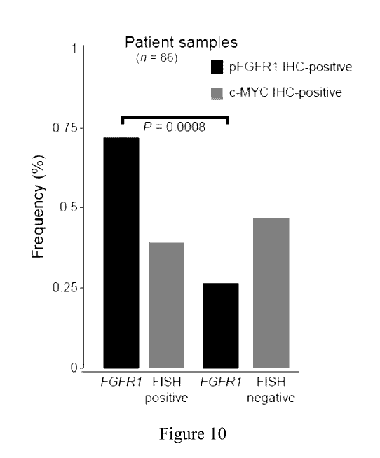

To extrapolate our finding that MYC expression levels dictate FGFR1 inhibitor sensitivity of FGFR/-amplified lung cancer to a larger panel of primary tumors, we screened a cohort of 306 squamous cell lung cancer biopsies for the presence of FGFR1 amplification by FISH. In this cohort 8p12 amplification occurred at a frequency of approximately 20%. A subcohort (n=86) enriched for FGFR1 amplification (78%) was further analyzed for p-FGFR1 and MYC expression by immunohistochemistry using a 4-tier scale by three independent observers (FIG. 10). We found strong membranous p-FGFR1 staining in this cohort. Only 26% of the amplified samples exhibited low scores of FGFR1 phosphorylation. In contrast, high levels of nuclear MYC staining did not segregate with amplification status of FGFR1 (frequency 40% in FGFR1 amp vs. 46% in FGFR1 non-amp; P=0.76). Thus, whereas most FGFR/-amplified squamous cell lung cancers exhibited FGFR1 phosphorylation, only a fraction of these cases also showed nuclear MYC expression. The finding that only a minority of FGFR/-amplified lung tumors are likely to respond to FGFR inhibition is consistent with the possibility that MYC expression predicts FGFR dependency in this cohort.

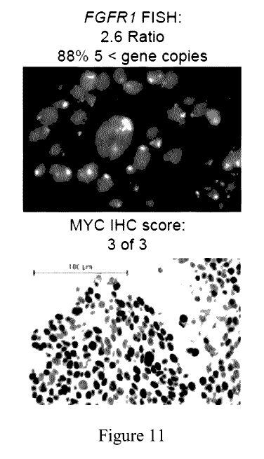

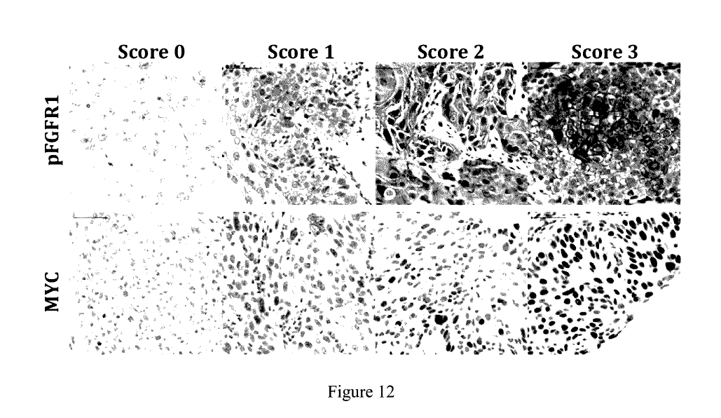

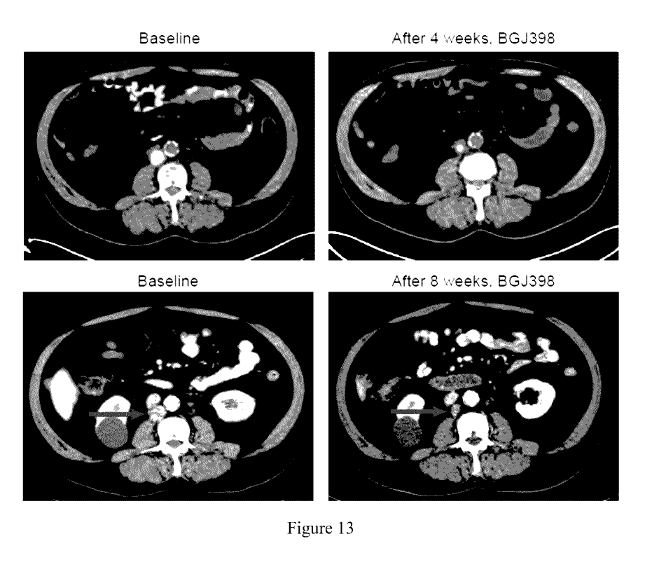

We identified a 65-year-old caucasian man with a 70-packper-year smoking history. The patient was diagnosed with stage IV squamous cell lung cancer and had been initially treated with two chemotherapy lines (a combination of carboplatinum and paclitaxel and docetaxel monotherapy). We observed amplification of FGFR1 (2.6 ratio-signals per cell on average, plus 88% of the cells harbored 5 or more gene copies) in the patient's tumor (FIG. 11). Immunohistochemical assessment revealed elevated expression levels of MYC with a score of 3 (FIG. 11 and FIG. 12). The patient agreed to treatment with BGJ398, a highly specific FGFR1 inhibitor, which was being evaluated in a first-in-humans trial at our center. After cardiac assessment and baseline thoracic computed tomography (CT), treatment with 100 mg BGJ398 was started. We observed a regression without cavitation of the tumor [CT scans after 4 and 8 weeks, partial response (PR) according to RECIST 1.1 criteria] and the patient experienced improvement of symptoms (FIG. 13). After 10 months of therapy, progressive disease (PD) was diagnosed in the kidney (PD as to RECIST1.1 criteria), so that BGJ398 treatment was stopped.

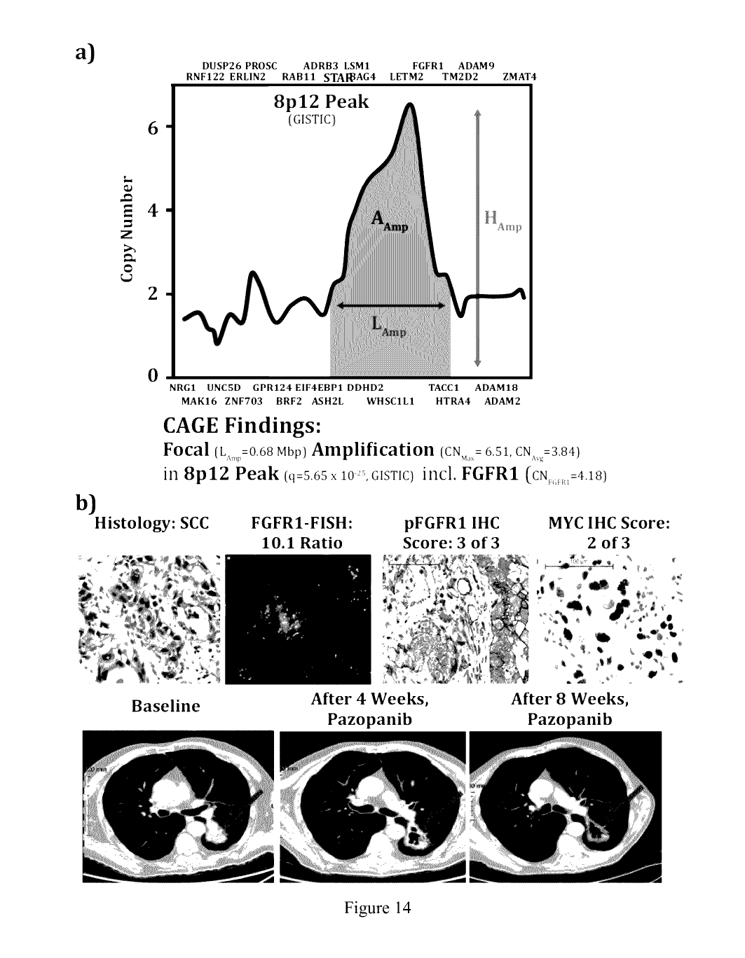

Another patient was diagnosed with metastatic squamous cell lung cancer and high-level amplification of FGFR1 (10.1 signals per cells on average) and high expression of MYC (FIG. 14). The FGFR1 amplification was highly focal, as determined by hybrid-capture-based massively parallel sequencing of 302 genes, enriched for the chromosomal region covering the 8p12 amplicon (FIG. 14a). The patient refused chemotherapy, but consented to off-label use of pazopanib, a multikinase inhibitor with weak activity against FGFR. After cardiac assessment and baseline thoracic CT, treatment with pazopanib 400 mg b.i.d. was started. Four weeks and eight weeks after the start of pazopanib, CT showed tumor regression with cavitation (FIG. 14b). Because of grade 2 fatigue, stomatitis, and gastrointestinal side effects, the patient decided to stop pazopanib after 6 months. At that time, no clinical or radiologic signs of tumor progression were present. We note that the inhibitory profile of pazopanib and the pseudocavernous response are also compatible with a predominant antiangiogenic effect. However, in light of our preclinical findings, we speculate that the patient's response might also be attributable to FGFR inhibition in the context of an MYC-expressing, FGFR1-amplified lung cancer.

All references cited herein are fully incorporated by reference. Having now fully described the invention, it will be understood by a person skilled in the art that the invention may be practiced within a wide and equivalent range of conditions, parameters and the liek, without affecting the spirit or scope of the invention or any embodiment thereof.

What is claimed is:

1. A method of treating cancer comprising administering an effective amount of an FGFR1 inhibitor to a cancer patient, wherein tumor cells or tissue obtained from the patient has been detected to exhibit (1) focal FGFR1 gene amplification or FGFR1 gene overexpression, and (2) MYC gene amplification or MYC gene overexpression.

2. The method of claim 1, wherein said FGFR1 inhibitor is chosen from antibodies selectively immunoreactive to FGFR1, small molecule inhibitors of FGFR1 kinase activity, FGF ligand traps, and antibodies selectively immunoreactive to FGF1.

3. The method of claim 1, wherein said tumor cells or tissue has been detected by IHC to overexpress FGFR1 and MYC.

4. The method of claim 1, wherein said tumor cells or tissue has been detected to harbor focal FGFR1 gene amplification and overexpress MYC protein.

5. The method of claim 1, wherein said cancer is lung cancer.

6. The method of claim 1, wherein said patient is diagnosed of squamous cell lung cancer.

7. A method of predicting a cancer patient's response to FGFR1 inhibitors, comprising:
detecting focal FGFR1 gene amplification or FGFR1 gene expression in a tumor cell or tissue obtained from a patient; and
detecting MYC gene amplification or MYC gene expression in said tumor cell or tissue or a second tumor cell or tissue from said patient, wherein the detection of both (1) focal FGFR1 gene amplification or increased FGFR1 gene expression, and (2) MYC gene amplification or increased MYC gene expression would indicate that said patient has an increased likelihood of response to FGFR1 inhibitors.

8. A diagnostic kit consisting essentially of, in a compartmentalized container:
a first nucleic acid primer or probe that hybridizes to the FGFR1 gene, or a first antibody selectively immunoreactive to FGFR1 protein; and
a second nucleic acid primer or probe that hybridizes to the MYC gene, or a second antibody selectively immunoreactive to c-MYC protein.
